(19) United States
(12) Patent Application Publication
(10) Pub. No.: US 2024/0245388 A1
(43) Pub. Date: Jul. 25, 2024

(54) METHOD FOR PROVIDING ULTRASOUND CATHETER WITH AN ACOUSTIC IMAGING MEDIUM

(71) Applicant: Boston Scientific Scimed, Inc., Maple Grove, MN (US)

(72) Inventor: Bryan Joseph Plunger, Shoreview, MN (US)

(73) Assignee: Boston Scientific Scimed, Inc., Maple Grove, MN (US)

(21) Appl. No.: 18/419,941

(22) Filed: Jan. 23, 2024

Related U.S. Application Data
(60) Provisional application No. 63/440,852, filed on Jan. 24, 2023.

(51) Int. Cl. A61B 8/00; A61B 8/08; A61B 8/12

(52) U.S. Cl. CPC A61B 8/445; A61B 8/0891; A61B 8/12

(57) ABSTRACT

An ultrasound catheter includes a distal port and a proximal port and is adapted to provide fluid communication between the distal port and the proximal port. The ultrasound catheter includes an ultrasound transducer. The ultrasound catheter may be advanced through a guide catheter that is positioned within a patient's vasculature and is fluidly coupled with the patient's blood. A vacuum may be applied to the proximal port, causing the patient's blood to be slowly pulled into the ultrasound catheter through the distal port such that any air near the ultrasound transducer is replaced with the patient's blood and the ultrasound transducer is surrounded by the patient's blood, which is an optimal imaging medium. The ultrasound transducer may be operated to obtain ultrasound images while applying the vacuum to the proximal port.

### CROSS-REFERENCE TO RELATED APPLICATIONS

This application claims the benefit of priority under 35 U.S.C. § 119 of U.S. Provisional Application No. 63/440,852, filed Jan. 24, 2023, the entire disclosure of which is hereby incorporated by reference.

### TECHNICAL FIELD

The present disclosure pertains to ultrasound catheters. More particularly, the present disclosure pertains to providing ultrasound catheters with an acoustic imaging medium.

### BACKGROUND

Ultrasound catheters utilize rotating ultrasound transducers in order to image internal structures such as portions of a patient's vasculature. Air within the ultrasound catheter can pose difficulties including poor acoustic imaging. In some cases, saline is used to flush the interior of an ultrasound catheter prior to use in order to remove any the air disposed within the ultrasound catheter. Unfortunately, in some cases some air may remain within the ultrasound catheter, even after a saline flush. There is an ongoing need to provide alternate methods for improving the performance of ultrasound catheters, including an ongoing need to provide alternate methods for providing improved acoustic medium within the ultrasound catheter.

### SUMMARY

This disclosure provides design, material, manufacturing method, and use alternatives for medical devices. An example may be found in a method for obtaining ultrasound images using an ultrasound catheter including an ultrasound transducer, a distal port and a proximal port, the ultrasound catheter adapted to provide fluid communication between the distal port and the proximal port. The method includes placing the ultrasound catheter with its distal port immersed in a patient's blood and applying a vacuum to the proximal port such that the patient's blood is slowly pulled into the ultrasound catheter, thereby displacing any air disposed near the ultrasound transducer. The method includes operating the ultrasound transducer to obtain ultrasound images while applying the vacuum to the proximal port.

Alternatively or additionally, the method may further include flushing the ultrasound catheter with saline prior to placing the ultrasound catheter with its distal port immersed in the patient's blood.

Alternatively or additionally, the method may further include flushing the ultrasound catheter with saline after operating the ultrasound transducer to obtain images.

Alternatively or additionally, applying a vacuum to the proximal port of the ultrasound catheter may include applying a vacuum of up to 100 millimeters mercury (100 mmHg).

Alternatively or additionally, applying a vacuum to the proximal port of the ultrasound catheter may include attaching a vacuum source to the proximal port.

Alternatively or additionally, applying a vacuum to the proximal port of the ultrasound catheter may include using a syringe attached to the proximal port to apply the vacuum.

Alternatively or additionally, positioning the ultrasound catheter with the distal port of the ultrasound catheter in fluid communication with the patient's blood may include advancing the ultrasound catheter within a guide catheter that is filled with the patient's blood.

Another example may be found in a method for preparing an ultrasound catheter for use in a patient, the ultrasound catheter including a distal port and a proximal port. The method includes positioning the ultrasound catheter with the distal port of the ultrasound catheter in fluid communication with a blood supply of the patient and applying a reduced pressure to the proximal port of the ultrasound catheter such that blood from the blood supply of the patient is slowly pulled into the ultrasound catheter, thereby expelling air from the ultrasound catheter.

Alternatively or additionally, applying a reduced pressure to the proximal port of the ultrasound catheter may include applying a vacuum of up to 100 millimeters mercury (100 mmHg).

Alternatively or additionally, applying a reduced pressure to the proximal port of the ultrasound catheter may include attaching a vacuum source to the proximal port.

Alternatively or additionally, applying a reduced pressure to the proximal port of the ultrasound catheter may include attaching a syringe to the proximal port, the syringe including a syringe body and a plunger disposed within the syringe body, and withdrawing the plunger relative to the syringe body to create a partial vacuum within the syringe body.

Alternatively or additionally, positioning the ultrasound catheter with the distal port of the ultrasound catheter in fluid communication with a blood supply of the patient may include advancing the ultrasound catheter within a guide catheter that is filled with the patient's blood.

Alternatively or additionally, the method may further include flushing the ultrasound catheter with saline prior to positioning the ultrasound catheter with the distal port of the ultrasound catheter in fluid communication with a blood supply of the patient.

Another example may be found in a method for using an ultrasound catheter having an ultrasound transducer, a distal port and a proximal port, the ultrasound catheter adapted to provide fluid communication between the distal port and the proximal port. The method includes advancing the ultrasound catheter through a guide catheter positioned within a patient's vasculature, the guide catheter fluidly coupled with the patient's blood, the ultrasound catheter advanced distally such that a distal port of the ultrasound catheter is immersed in the patient's blood within the guide catheter. A vacuum is applied to the proximal port such that the patient's blood is slowly pulled into the ultrasound catheter such that any air near the ultrasound transducer is replaced with the patient's blood and the ultrasound transducer is surrounded by the patient's blood. The method includes operating the ultrasound transducer to obtain ultrasound images while applying the vacuum to the proximal port.

Alternatively or additionally, the method may further include flushing the ultrasound catheter with saline prior to advancing the ultrasound catheter through the guide catheter.

Alternatively or additionally, the method may further include withdrawing the ultrasound catheter from the guide catheter, flushing the ultrasound catheter with saline to flush blood out of the ultrasound catheter, and subsequently re-advancing the ultrasound catheter through the guide catheter.

Alternatively or additionally, applying a vacuum to the proximal port of the ultrasound catheter may include applying a vacuum of up to 100 millimeters mercury (100 mmHg).

Alternatively or additionally, applying a vacuum to the proximal port of the ultrasound catheter may include attaching a vacuum source to the proximal port.

Alternatively or additionally, applying a vacuum to the proximal port of the ultrasound catheter may include using a syringe attached to the proximal port to apply the vacuum.

Alternatively or additionally, surrounding the ultrasound transducer with the patient's blood may minimize acoustic impedance changes in an ultrasound beam path emanating from the ultrasound transducer.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an illustrative medical device;

FIG. 2 is a side view of a portion of an illustrative medical device;

FIG. 3 is a side view of an illustrative IVUS catheter; and

FIG. 4 is a side view of an illustrative IVUS catheter.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

As used herein, the term “ultrasonic energy” is used broadly, includes its ordinary meaning, and further includes mechanical energy transferred through pressure or compression waves with a frequency between about 1 MHz to about 80 MHz. As used herein, the term “catheter” is used broadly, includes its ordinary meaning, and further includes an elongate flexible tube configured to be inserted into the body of a patient, such as into a body part, cavity, duct or vessel. As used herein, the term “end” is used broadly, includes its ordinary meaning, and further encompasses a region generally, such that “proximal end” includes “proximal region”, and “distal end” includes “distal region”.

As used herein, the term “ultrasound radiating member” refers to any apparatus capable of producing ultrasonic energy. For example, in one embodiment, an ultrasound radiating member comprises an ultrasonic transducer, which converts electrical energy into ultrasonic energy. A suitable example of an ultrasonic transducer for generating ultrasonic energy from electrical energy includes, but is not limited to, piezoelectric ceramic oscillators. Piezoelectric ceramics may include a crystalline material, such as quartz, that changes shape when an electrical voltage is applied to the material. This change in shape, made oscillatory by an oscillating driving signal, creates ultrasonic sound waves. In other embodiments, ultrasonic energy can be generated by an ultrasonic transducer that is remote from the ultrasound radiating member, and the ultrasonic energy can be transmitted, via, for example, a wire that is coupled to the ultrasound radiating member.

As will be described below, the ultrasound catheter can include two or more ultrasound radiating members positioned therein. Such ultrasound radiating members can include a transducer (e.g., a PZT transducer), which is configured to convert electrical energy into ultrasonic energy. In such embodiments, the PZT transducer is excited by specific electrical parameters (herein “power parameters” that cause it to vibrate in a way that generates ultrasonic energy).

In some cases, an ultrasound radiating member may be operated in a pulsed mode. For example, the time average electrical power supplied to an ultrasound radiating member may be between about 0.001 watts and about 5 watts and may be between about 0.05 watts and about 3 watts. In some embodiments, the time average electrical power over treatment time may be about 0.45 watts or 1.2 watts. The duty cycle may be between about 0.01% and about 90%. In some instances, the duty cycle may be between about 0.1% and about 50%. In some embodiments, the duty ratio may be about 7.5%, 15% or a variation between 1% and 30%. The pulse average electrical power may be between about 0.01 watts and about 40 watts and may be between about 0.1 watts and 20 watts. In some embodiments, the pulse averaged electrical power may be about 4 watts, 8 watts, 16 watts, or a variation of 1 to 16 watts. The amplitude, pulse width, pulse repetition frequency, average acoustic pressure or any combination of these parameters may be constant or varied during each pulse or over a set of pulses. In a non-linear application of acoustic parameters the above ranges can change significantly. Accordingly, the overall time average electrical power over treatment time may stay constant throughout the treatment or may vary in real time during the treatment. the same but not real-time average power.

In some cases, an ultrasound radiating member may include piezoelectric material and may have any of a variety of different shapes. An ultrasound radiating member may include a cylindrical or rectangular or disc shaped. An ultrasound radiating member may be a single transducer or may be designed as an array of small transducers such as a one-dimensional array or a two-dimensional array.

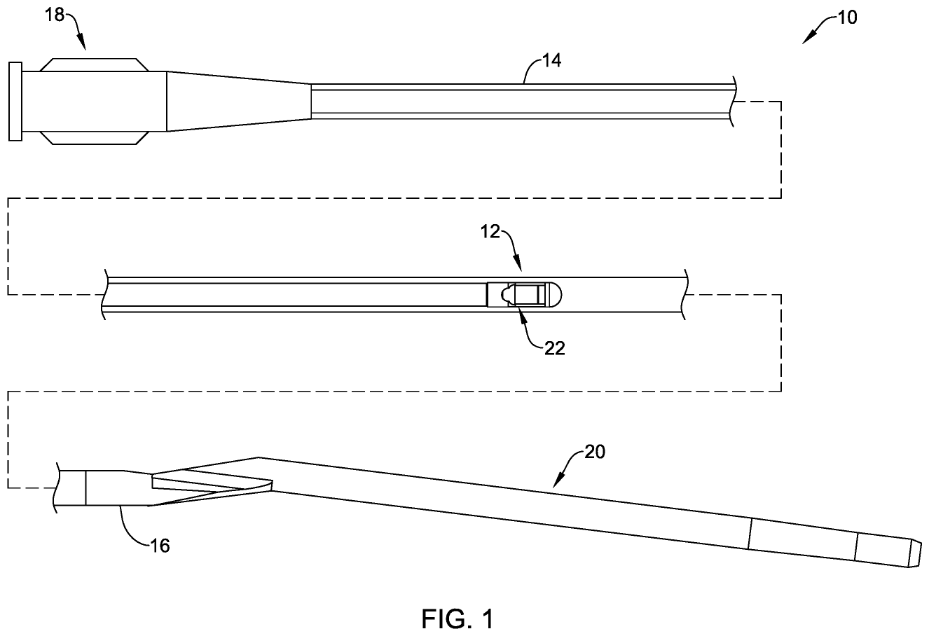

FIG. 1 is a side view of an example medical device 10. In at least some instances, the medical device 10 takes the form of an imaging medical device. For example, the medical device 10 may be an intravascular ultrasound (IVUS) device that may be used to image a blood vessel. The structure/form of the medical device 10 can vary. In some instances, the medical device 10 may include an elongate shaft 12 having a proximal end region 14 and a distal end region 16. A hub 18 may be coupled to or otherwise disposed adjacent to the proximal end region 14. A tip member 20 may be coupled to or otherwise disposed adjacent to the distal end region 16. The tip member 20 may include a guidewire lumen, an atraumatic distal end, one or more radiopaque markers, and/or other features. An imaging assembly 22 may be disposed within the shaft 12. In general, the imaging assembly may be used to capture/generate images of a blood vessel. In some instances, the medical device may include devices and/or features similar to those disclosed in U.S. Patent Application Pub. No. US 2012/0059241 and U.S. Patent Application Pub. No. US 2017/0164925, the entire disclosures of which are herein incorporated by reference. In at least some instances, the medical device 10 may resemble and/or include features that resemble the OPTICROSS™ Imaging Catheter, commercially available from BOSTON SCIENTIFIC, Marlborough, MA.

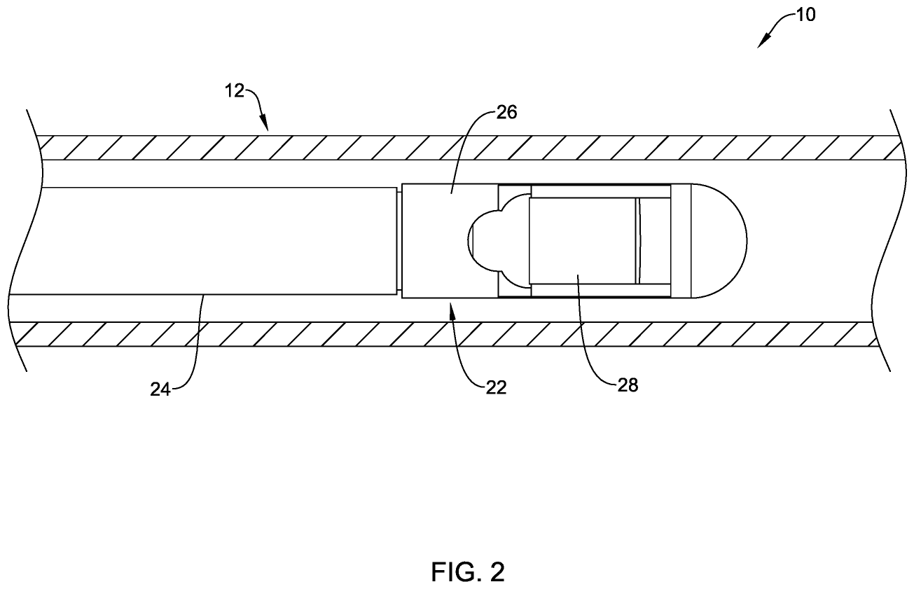

The imaging assembly 22 may include a drive cable or shaft 24, a housing 26, and an imaging member or transducer 28 coupled to the drive cable 24 and/or housing 26 as shown in FIG. 2. In at least some instances, the transducer 28 includes an ultrasound transducer. Other transducers are also contemplated. The transducer 28 may be rotatable and/or axially translatable relative to the shaft 12. For example, the drive cable 24 may be rotated and/or translated in order to rotate and/or translate the transducer 28 (and the housing 26).

In some instances, the medical device 10 may be initially flushed with saline in order to remove air from within the elongate shaft 12. While flushing the medical device 10 with saline may displace most of the air within the elongate shaft 12, it will be appreciated that in some instances some air may remain. If any of the remaining air ends up near the imaging assembly 22, the images provided by the imaging assembly 22 may be degraded if there is any air within the medical device 10, particularly if the air is near the imaging assembly 22 such that an ultrasound beam path emanating from the imaging assembly 22 passes through air. In some instances, rotation of the drive cable 24 may cause air bubbles within the medical device 10 to translate through the elongate shaft 12, and thus may reach the imaging assembly 22. Accordingly, it is desirable to remove all air from the medical device 10, and particularly from the region around the imaging assembly 22.

As noted, a saline flush may be used prior to use to remove as much air as possible. This can result in the imaging assembly 22 being bathed in saline. While saline is a better medium than air, the acoustic impedance of saline does not exactly match that of blood. In some cases, it may be beneficial if the medium immediately surrounding the imaging assembly 22 provides an acoustic impedance that is as close to that of the blood exterior to the medical device 10 as this will minimize acoustic impedance changes within the ultrasound beam path. Accordingly, in some cases the patient's blood may be drawn into the distal end region 16 of the medical device 10 in order to displace any less than optimal fluids around the imaging assembly 22 such as air and/or saline and to replace those fluids with the patient's blood. It will be appreciated that the acoustic impedance of the patient's blood within the medical device 10 will match that of the patient's blood outside of the medical device 10, apart from any changes in acoustic impedance as a result of the ultrasound beam path passing through the materials forming the elongate shaft 12, for example.

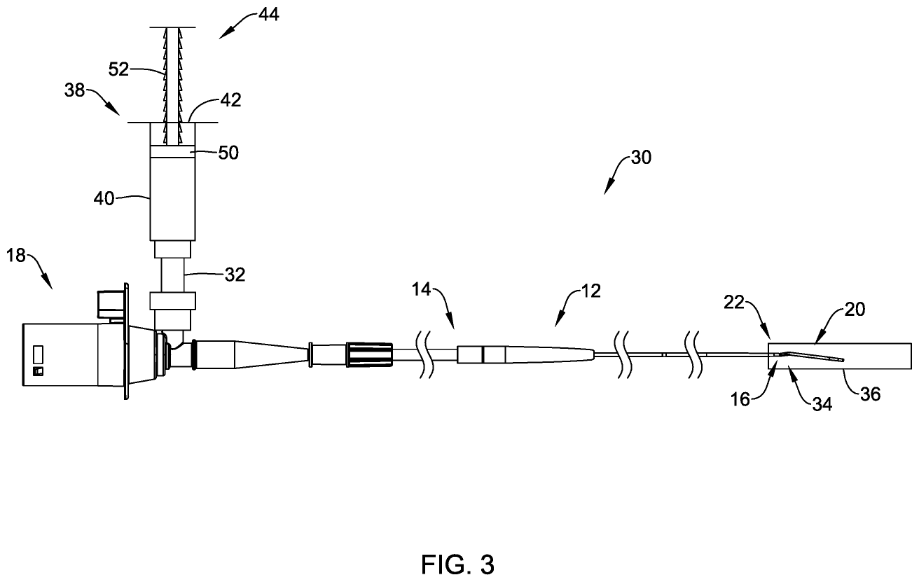

FIG. 3 is a schematic view of an illustrative IVUS catheter 30 that is adapted to allow for use of the patient's blood as a medium surrounding the imaging assembly 22. The IVUS catheter 30 may be considered as being an example of the medical device 10 described with respect to FIG. 1. The IVUS catheter 30 includes some of the same features as the medical device 10, including the elongate shaft 12 having the proximal end region 14 and the distal end region 16. The hub 18 may be coupled to or otherwise disposed adjacent to the proximal end region 14. It will be appreciated that the hub 18 may take any of a variety of forms, including those shown in FIG. 1 and in FIG. 3. Other forms for the hub 18 are also contemplated. The tip member 20 may be coupled to or otherwise disposed adjacent to the distal end region 16. The tip member 20 may include a guidewire lumen, an atraumatic distal end, one or more radiopaque markers, and/or other features. An imaging assembly 22 (not visible here) may be disposed within the shaft 12 and may include the features discussed with respect to FIG. 2, such as a drive cable, housing and imaging member or transducer that is coupled to the drive cable and/or the housing. The transducer may include an ultrasound transducer. Other transducers are also contemplated. The transducer may be rotatable and/or axially translatable relative to the shaft 12. For example, the drive cable may be rotated and/or translated in order to rotate and/or translate the transducer (and the housing).

The IVUS catheter 30 also includes features that allow for use of the patient's blood in providing an optimal acoustic medium around the imaging assembly 22. As shown, the IVUS catheter 30 includes a proximal port 32 and a distal port 34. The proximal port 32 is disposed within or adjacent to the hub 18. As shown, the proximal port 32 takes the form of a luer fitting, but this is not required in all cases. In some cases, the proximal port 32 may be adapted to permit fluids such as air and saline, and even blood, to exit the proximal port 32 but may not permit fluids to pass distally through the proximal port 32. In some cases, the proximal port 32 may be reversible, such as to selectively allow fluids to be pulled proximally through the IVUS catheter 30 and out of the proximal port 32, and to allow fluids such as saline to be pumped distally through the proximal port 32 when flushing the IVUS catheter 30 with saline. In some cases, the IVUS catheter 30 may be flushed with saline by placing the distal port 34 into fluid communication with a source of saline, and withdrawing fluids proximally through the proximal port 32. The distal port 34 may be located within the distal end region 16 of the elongate shaft 12. In some cases, the distal port 34 may be located near where the tip member 20 attaches to the distal end region 16 of the elongate shaft 12. These are just examples.

In use, the distal port 34 may be placed into a source of the patient's blood. In some cases, the distal port 34 may be advanced into a schematically-shown guide catheter 36. By applying a vacuum or reduced pressure at the proximal port 32, blood from within the guide catheter 36 may be withdrawn proximally through the elongate shaft 12. Thus, the blood pulled into the elongate shaft 12 will displace any air (or saline) that is disposed at or around the imaging assembly 22. In some cases, this may improve the quality of any ultrasound images obtained using the IVUS catheter 30 because the acoustic impedance of the blood surrounding the imaging assembly 22 inside the elongate shaft 12 will match the acoustic impedance of the blood outside of the elongate shaft 12. This may also improve the quality of any ultrasound images obtained using the IVUS catheter 30 because pulling blood into and through the elongate shaft 12 will reduce or even eliminate any air bubbles that may otherwise be present.

A reduced pressure or vacuum may be applied to the proximal port 32 in any of a variety of manners. In some cases, as shown, a syringe 38 may be releasably secured to the proximal port 32. In some cases, the syringe 38 may include a distal end adapted to be threadedly engaged with a luer lock. The syringe 38 includes a body 40 having a syringe top 42. A plunger 44 extends through the syringe top 42. The plunger 44 includes a handle 46, a plunger 48 extending away from the handle 46, and an elastic member 50 that fits within the body 40 such that pulling up (in the illustrated orientation) on the handle 46, and thus the plunger 48, causes the elastic member 50 to move in an upward direction. The elastic member 50 moves upward as well, causing a reduced pressure in the body 40 below the elastic member 50. If the handle 46 and thus the plunger 48 were to move in a downward direction, the elastic member 50 would also move in a downward direction, causing an increased pressure in the body 40 below the elastic member 50.

In some cases, the plunger 48 may be adapted to interact with the syringe top 42 in a way that allows the handle 46 and the plunger 48 (and hence the elastic member 50) to be pulled in an upward (in the illustrated orientation) direction but does not allow the handle 46 and the plunger 48 (and the elastic member 50) to move in a downward direction. This may allow the syringe 38 to be used to apply a negative pressure or vacuum to the proximal port 32 because once the plunger 48 has been pulled in an upward direction, the plunger 48 is not able to move back down. As an example, the plunger 48 may include a serrated pattern 52 that interacts with a corresponding aperture in the syringe tip 42. As another example, in some cases the syringe 38 may include a locking mechanism by which the plunger 48 may be pulled upward and then rotated to lock the plunger 48 in position.

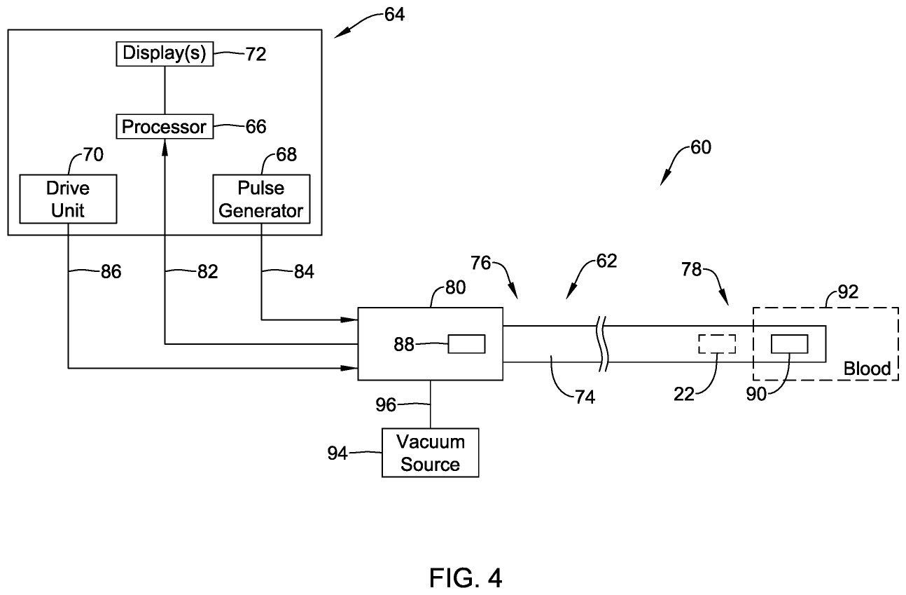

FIG. 4 is a schematic view of an illustrative IVUS imaging system 60 that is adapted to allow for use of the patient's blood as a medium surrounding the imaging assembly 22. The IVUS imaging system 60 includes an IVUS catheter 62 that is adapted to be coupled with a control module 64. The IVUS catheter 62 may be considered as being an example of the medical device 10 described with respect to FIG. 1. The control module 64 may include, for example, a processor 66, a pulse generator 68, a drive unit 70, and one or more displays 72. In at least some embodiments, the pulse generator 68 forms electric signals that are input to the imaging assembly 22 disposed in the IVUS catheter 62. In at least some embodiments, electric signals transmitted from the imaging assembly 22 may be input to the processor 66 for processing. The processed electric signals from the imaging assembly 22 may be displayed as one or more images on the one or more displays 72. In at least some embodiments, mechanical energy from the drive unit 70 is used to drive an imaging core disposed in the IVUS catheter 62.

The processor 66 may also be used to control the functioning of one or more of the other components of the control module 64. For example, the processor 66 may be used to control at least one of the frequency or duration of the electrical signals transmitted from the pulse generator 68, the rotation rate of the imaging core (by the drive unit 70, the velocity or length of the pullback of the imaging core by the drive unit 70, or one or more properties of one or more images formed on the one or more displays 72.

The IVUS catheter 62 includes an elongate shaft 74 that extends from a proximal end region 76 to a distal end region 78. A hub 80 is secured to the proximal end region 76. The hub 80 may be adapted to receive one or more wires or cables emanating from the control module 64, such as a cable 82 carrying electrical signals from the hub 80 to the processor 66. A cable 84 carries electrical signals from the pulse generator 68 to the hub 80. A drive cable 86 may be driven into rotation by the drive unit 70, and may be used to drive into rotation a drive cable extending through the elongate shaft 74 (akin to the drive cable 24 shown in FIG. 2).

The IVUS catheter 62 includes a proximal port 88 that is located within the proximal end region 76 of the elongate shaft 74, or even within the hub 80. A distal port 90 is located within the distal end region 78 of the elongate shaft 74. While not expressly shown, it will be appreciated that the proximal port 88 is adapted to be in fluid communication with the distal port 90. This means that a fluid pumped into the proximal port 88 may pass through the elongate shaft 74 and exit the distal port 90. A fluid drawn into the distal port 90 may pass through the elongate shaft 74 and exit the proximal port 88, for example.

In use, the IVUS catheter 62 may be positioned such that the distal end region 78 of the elongate shaft 74, including the distal port 90, is disposed within a blood source 92. In some cases, the blood source 92 may represent a guide catheter that has been advanced through the patient's vasculature to a location of interest within the vasculature. A vacuum source 94 may be coupled with the proximal port 88 via a connection 96 in order to apply a reduced pressure or vacuum to the proximal port 88. This causes a reduced pressure within the elongate shaft 74, which in turn causes blood to be pulled into the elongate shaft 74 via the distal port 90. As the blood enters the elongate shaft 74, the blood displaces any air bubbles or other fluids such as saline that may be present near or around the imaging assembly 22.

The vacuum source 94 may be a syringe such as the syringe 38 shown in FIG. 3. In some cases, the vacuum source 94 may be a vacuum source within the room, such as may be available in a treatment room. In some cases, the vacuum source 94 may be adapted to provide a vacuum or reduced pressure of about 100 mmHg (millimeters mercury). Other levels of vacuum are also contemplated. In some instances, a greater vacuum may be provided if there is a desire to more quickly pull blood into the elongate shaft 74 and thus provide improved performance more quickly. In some instances, a lesser vacuum may be provided if there is a desire to more slowly pull blood into the elongate shaft 74, thereby potentially increasing a procedure time duration by not filling the elongate shaft 74 with blood too quickly.

In some cases, a method for obtaining ultrasound images using an ultrasound catheter including an ultrasound transducer includes placing the IVUS catheter 62 with its distal port 90 immersed in a patient's blood 92. In some cases, positioning the IVUS catheter 62 with the distal port 90 of the IVUS catheter 62 in fluid communication with the patient's blood 92 may include advancing the ultra IVUS catheter 62 within a guide catheter that is filled with the patient's blood. A vacuum may be applied to the proximal port 88 such that the patient's blood is slowly pulled into the IVUS catheter 62, thereby displacing any air disposed near the ultrasound transducer. The ultrasound transducer may be operated to obtain ultrasound images while applying the vacuum to the proximal port 88.

In some cases, while not required, the method may further include flushing the IVUS catheter 62 with saline prior to placing the IVUS catheter 62 with its distal port 90 immersed in the patient's blood 92. In some cases, while not required, the method may further include flushing the IVUS catheter 62 with saline after operating the ultrasound transducer to obtain images.

In some instances, applying a vacuum to the proximal port 88 of the IVUS catheter 62 may include applying a vacuum of up to 100 millimeters mercury (100 mmHg). As an example, applying a vacuum to the proximal port 88 of the IVUS catheter 62 may include attaching the vacuum source 94 to the proximal port 88. As an example, applying a vacuum to the proximal port 88 of the IVUS catheter 62 may include using a syringe (such as the syringe 38) attached to the proximal port 88 to apply the vacuum.

In some cases, a method for preparing the IVUS catheter 62 for use in a patient may include positioning the IVUS catheter 62 with the distal port 90 of the IVUS catheter 62 in fluid communication with a blood supply of the patient (such as the blood 92). A reduced pressure may be applied to the proximal port 88 of the IVUS catheter 62 such that blood from the blood supply of the patient is slowly pulled into the IVUS catheter 62, thereby expelling air from the IVUS catheter 62. In some instances, positioning the IVUS catheter 62 with the distal port 90 of the IVUS catheter 62 in fluid communication with a blood supply of the patient may include advancing the IVUS catheter 62 within a guide catheter that is filled with the patient's blood. In some cases, the method may further include flushing the IVUS catheter 62 with saline prior to positioning the IVUS catheter 62 with the distal port 90 of the IVUS catheter 62 in fluid communication with a blood supply of the patient.

In some instances, applying a reduced pressure to the proximal port 88 of the IVUS catheter 62 may include applying a vacuum of up to 100 millimeters mercury (100 mmHg). As an example, applying a reduced pressure to the proximal port 88 of the IVUS catheter 62 may include attaching the vacuum source 94 to the proximal port 88. As another example, applying a reduced pressure to the proximal port 88 of the IVUS catheter 62 may include attaching a syringe (such as the syringe 38) to the proximal port 88, the syringe including a syringe body and a plunger disposed within the syringe body, and withdrawing the plunger relative to the syringe body to create a partial vacuum within the syringe body.

In some cases, a method for using the IVUS catheter 62 may include advancing the IVUS catheter 62 through a guide catheter positioned within a patient's vasculature, the guide catheter fluidly coupled with the patient's blood, the IVUS catheter 62 advanced distally such that a distal port of the IVUS catheter 62 is immersed in the patient's blood within the guide catheter. A vacuum is applied to the proximal port 88 such that the patient's blood is slowly pulled into the IVUS catheter 62 such that any air near the ultrasound transducer is replaced with the patient's blood and the ultrasound transducer is surrounded by the patient's blood. The ultrasound transducer is operated to obtain ultrasound images while applying vacuum to the proximal port 88. In some cases, surrounding the ultrasound transducer with the patient's blood minimizes acoustic impedance changes in an ultrasound beam path emanating from the ultrasound transducer.

In some cases, the method may further include flushing the IVUS catheter 62 with saline prior to advancing the IVUS catheter 62 through the guide catheter. In some cases, the method may further include withdrawing the IVUS catheter 62 from the guide catheter, flushing the IVUS catheter 62 with saline to flush blood out of the IVUS catheter 62, and subsequently re-advancing the IVUS catheter 62 through the guide catheter. This may be done when there is a desire to take a break from use of the IVUS catheter 62, but subsequent reuse of the IVUS catheter 62 with the same patient is contemplated.

In some instances, applying a reduced pressure to the proximal port 88 of the IVUS catheter 62 may include applying a vacuum of up to 100 millimeters mercury (100 mmHg). As an example, applying a reduced pressure to the proximal port 88 of the IVUS catheter 62 may include attaching the vacuum source 94 to the proximal port 88. As another example, applying a reduced pressure to the proximal port 88 of the IVUS catheter 62 may include attaching a syringe (such as the syringe 38) to the proximal port 88.

The materials that can be used for the various components of the devices described herein may include those commonly associated with medical devices. The devices and components thereof described herein may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the devices described herein may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the devices described herein in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the devices described herein to achieve the same result.

In some embodiments, a degree of Magnetic Resonance Imaging (MRI) compatibility is imparted into the devices described herein. For example, the devices described herein, or portions thereof, may be made of a material that does not substantially distort the image and create substantial artifacts (e.g., gaps in the image). Certain ferromagnetic materials, for example, may not be suitable because they may create artifacts in an MRI image. The devices described herein, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A method for obtaining ultrasound images using an ultrasound catheter including an ultrasound transducer, a distal port and a proximal port, the ultrasound catheter adapted to provide fluid communication between the distal port and the proximal port, the method comprising:
placing the ultrasound catheter with its distal port immersed in a patient's blood;
applying a vacuum to the proximal port such that the patient's blood is slowly pulled into the ultrasound catheter, thereby displacing any air disposed near the ultrasound transducer; and
while applying the vacuum to the proximal port, operating the ultrasound transducer to obtain ultrasound images.

2. The method of claim 1, further comprising flushing the ultrasound catheter with saline prior to placing the ultrasound catheter with its distal port immersed in the patient's blood.

3. The method of claim 1, further comprising flushing the ultrasound catheter with saline after operating the ultrasound transducer to obtain images.

4. The method of claim 1, wherein applying a vacuum to the proximal port of the ultrasound catheter comprises applying a vacuum of up to 100 millimeters mercury (100 mmHg).

5. The method of claim 1, wherein applying a vacuum to the proximal port of the ultrasound catheter comprises attaching a vacuum source to the proximal port.

6. The method of claim 1, wherein applying a vacuum to the proximal port of the ultrasound catheter comprises using a syringe attached to the proximal port to apply the vacuum.

7. The method of claim 1, wherein positioning the ultrasound catheter with the distal port of the ultrasound catheter in fluid communication with the patient's blood comprises advancing the ultrasound catheter within a guide catheter that is filled with the patient's blood.

8. A method for preparing an ultrasound catheter for use in a patient, the ultrasound catheter including a distal port and a proximal port, the method comprising:
positioning the ultrasound catheter with the distal port of the ultrasound catheter in fluid communication with a blood supply of the patient; and
applying a reduced pressure to the proximal port of the ultrasound catheter such that blood from the blood supply of the patient is slowly pulled into the ultrasound catheter, thereby expelling air from the ultrasound catheter.

9. The method of claim 8, wherein applying a reduced pressure to the proximal port of the ultrasound catheter comprises applying a vacuum of up to 100 millimeters mercury (100 mmHg).

10. The method of claim 8, wherein applying a reduced pressure to the proximal port of the ultrasound catheter comprises attaching a vacuum source to the proximal port.

11. The method of claim 8, wherein applying a reduced pressure to the proximal port of the ultrasound catheter comprises:
attaching a syringe to the proximal port, the syringe including a syringe body and a plunger disposed within the syringe body; and
withdrawing the plunger relative to the syringe body to create a partial vacuum within the syringe body.

12. The method of claim 8, wherein positioning the ultrasound catheter with the distal port of the ultrasound catheter in fluid communication with a blood supply of the patient comprises advancing the ultrasound catheter within a guide catheter that is filled with the patient's blood.

13. The method of claim 8, further comprising flushing the ultrasound catheter with saline prior to positioning the ultrasound catheter with the distal port of the ultrasound catheter in fluid communication with a blood supply of the patient.

14. A method for using an ultrasound catheter having an ultrasound transducer, a distal port and a proximal port, the ultrasound catheter adapted to provide fluid communication between the distal port and the proximal port, the method comprising:
advancing the ultrasound catheter through a guide catheter positioned within a patient's vasculature, the guide catheter fluidly coupled with the patient's blood, the ultrasound catheter advanced distally such that a distal port of the ultrasound catheter is immersed in the patient's blood within the guide catheter;
applying a vacuum to the proximal port such that the patient's blood is slowly pulled into the ultrasound catheter such that any air near the ultrasound transducer is replaced with the patient's blood and the ultrasound transducer is surrounded by the patient's blood; and
while applying the vacuum to the proximal port, operating the ultrasound transducer to obtain ultrasound images.

15. The method of claim 14, further comprising flushing the ultrasound catheter with saline prior to advancing the ultrasound catheter through the guide catheter.

16. The method of claim 14, further comprising:
withdrawing the ultrasound catheter from the guide catheter;
flushing the ultrasound catheter with saline to flush blood out of the ultrasound catheter; and
subsequently re-advancing the ultrasound catheter through the guide catheter.

17. The method of claim 14, wherein applying a vacuum to the proximal port of the ultrasound catheter comprises applying a vacuum of up to 100 millimeters mercury (100 mmHg).

18. The method of claim 14, wherein applying a vacuum to the proximal port of the ultrasound catheter comprises attaching a vacuum source to the proximal port.

19. The method of claim 14, wherein applying a vacuum to the proximal port of the ultrasound catheter comprises using a syringe attached to the proximal port to apply the vacuum.

20. The method of claim 14, wherein surrounding the ultrasound transducer with the patient's blood minimizes acoustic impedance changes in an ultrasound beam path emanating from the ultrasound transducer.
